(19) United States
(12) Patent Application Publication
(10) Pub. No.: US 2018/0021268 A1
(43) Pub. Date: Jan. 25, 2018

(54) WEIGHT LOSS/REDUCTION OF VISCERAL FAT COMPOUND AND METHOD OF EXTRACTION AND PREPARATION

(71) Applicant: JBSL-USA, INCORPORATED, Walnut Greek, CA (US)

(72) Inventor: Vincent Hackel, Walnut Creek, CA (US)

(73) Assignee: JBSL-USA, INCORPORATED, Walnut Creek, CA (US)

(21) Appl. No.: 15/650,820

(22) Filed: Jul. 14, 2017

Related U.S. Application Data
(60) Provisional application No. 62/363,133, filed on Jul. 15, 2016.

(51) Int. Cl. A61K 31/12; A61K 31/37; A61K 36/232

(52) U.S. Cl. CPC A61K 31/12; A61K 36/232; A61K 31/37; A61K 2236/13; A61K 2236/15; A61K 2236/17; A61K 2236/31; A61K 2236/51

(57) ABSTRACT

A composition for weight loss comprising at least 8% total chalcones comprising 4-hydroxyderricin and xanthoangelol and coumarins. A composition for reduction of visceral fat comprising at least 8% total chalcones comprising 4-hydroxyderricin and xanthoangelol and coumarins. A method to isolate and purify chalcones.

### CROSS-REFERENCE TO RELATED APPLICATIONS

This application claims the benefit of U.S. Provisional Patent Application No. 62/363,133 titled “Weight Loss/Reduction of Visceral Fat Compound and Method of Extraction and Preparation,” filed Jul. 15, 2016, the contents of which are incorporated herein by reference in their entirety.

### BACKGROUND OF THE INVENTION

Angelica keiskei Koidzumi, or Ashitaba, is a perennial plant belonging to the family Umbelliferae. Ashitaba has been consumed for many hundreds of years by the inhabitants of Seven Islands of Izu, and is believed to induce a variety of health-promoting effects. Examples of these health-promoting effects include an anti-bacterial effect, an anti-tumor effect, a suppressive action for gastric acid secretion, an anti-cancerous effect, and an enhancing effect for nerve growth factor production and hepatocyte growth factor production.

It is known that Ashitaba contains two types of chalcones as its main components: xanthoangelol and 4-hydroxyderricin. Various bioactive effects of chalcones have been reported, e.g., anti-bacterial activity, vasodilation action, anti-tumor activity, inhibition of gastric acid secretion, anti-inflammatory effects, and effects on blood coagulation. Recently, effects of chalcones such as inhibition of the proliferation of tumor cells and induction of apoptosis have been reported. In addition, some other functions of chalcones have been suggested e.g., reduction of cellulite, and the promotion of fat or adipose tissue metabolism and the inhibition of fat or adipose tissue accumulation in animals.

Coumarin is an organic compound classified as a benzopyrone. It can be made in a laboratory but is also found naturally in many plants such as the tonka bean, vanilla grass, sweet woodruff, mullein, sweet grass, cassia cinnamon, sweet-clover, deertongue, strawberries, black currants, apricots, cherries, and Justicia pectoralis.

Current methods of purification or isolation of chalcones and coumarins from whole Ashitaba leaves do not produce a high concentration of chalcones and coumarins in the end product. Accordingly, there is a need for methods to produce isolated chalcones and coumarin compounds that can be used in compositions, particularly compositions used to improve weight loss/reduction of visceral fat. The present invention satisfies this need.

### SUMMARY OF THE INVENTION

In one aspect of the invention, the invention comprises a composition for weight loss comprising at least 8% total chalcones comprising 4-hydroxyderricin and xanthoangelol, and coumarins.

Optionally, the coumarins are selected from a group comprising furanocoumarin type coumarins and dihydropyrano-coumarins.

Additionally, the furanocoumarins are selected from a group comprising xanthotoxin (psoralen), isopimpinellin, bergapten, and imperatorin.

Optionally, the dihydropyrano-coumarins are selected from a group comprising laserpitin, isolaserpitin, and selinidin.

Optionally, the amount of coumarins can comprise approximately 35 mg/g to 45 mg/g.

In a second aspect of the invention, the invention comprises a composition for reduction of visceral fat comprising at least 8% total chalcones comprising 4-hydroxyderricin and xanthoangelol, and coumarins.

Optionally, the coumarins are selected from a group comprising furanocoumarin type coumarins and dihydropyrano-coumarins.

Additionally, the furanocoumarins are selected from a group comprising xanthotoxin (psoralen), isopimpinellin, bergapten, and imperatorin.

Optionally, the dihydropyrano-coumarins are selected from a group comprising laserpitin, isolaserpitin, and selinidin.

Optionally, the amount of coumarins can comprise approximately 35 mg/g to 45 mg/g.

In a third aspect of the invention, the invention comprises a method to isolate and purify chalcones.

In a fourth aspect, the invention comprises a method for inducing weight loss comprising administration of an effective amount of chalcone powder obtained by a method to isolate and purify chalcones.

In a fifth aspect, the invention comprises a method for inducing reduction of visceral fat comprising administration of an effective amount of chalcone powder obtained by a method to isolate and purify chalcones.

Optionally, the method of isolation and purifying chalcones comprises the steps of a) cutting a stem of an Ashitaba plant, b) harvesting sap from the cut stem, c) pasteurizing the sap, d) mixing the pasteurized sap with cyclodextrin to produce a mixture, e) sterilizing the mixture, f) freeze-drying the mixture, and g) pulverizing the mixture.

Additionally, the step of pasteurizing the sap comprises heating the sap at 90° C. for 30 minutes. The step of sterilizing the mixture comprises heating the mixture at 121° C. for 15 minutes. Also, the step of pulverizing comprises passing the mixture through a 100 mesh.

### DRAWINGS

These and other features, aspects and advantages of the present invention will become better understood with reference to the following description, appended claims, and accompanying drawings where:

FIG. 1 is a flow chart showing a method of purification of Ashitaba chalcone powder, according to one embodiment of the invention as described in Example 1;

FIG. 2 depicts a graph showing the amount of weight gained by different mice after consuming the high fat, high cholesterol diet of stage one of Example 2;

FIG. 3 depicts a graph showing the increase in visceral fat of different mice after consuming the high fat, high cholesterol diet of stage one of Example 2;

FIG. 4 depicts a graph showing the liver triglyceride content of different mice after consuming the high fat, high cholesterol diet of stage one of Example 2;

FIG. 5 depicts a graph showing the amount of weight gained by different mice after consuming the high fat, high sugar diet of stage two of Example 2;

FIG. 6 depicts a graph showing the increase in visceral fat of different mice after consuming the high fat, high sugar diet of stage two of Example 2;

FIG. 7 depicts a graph showing the liver triglyceride content of different mice after consuming the high fat, high sugar diet of stage two of Example 2;

FIG. 8 depicts a graph showing the amount of weight gained by different mice after consuming the high fat diet of stage three of Example 2;

FIG. 9 depicts a graph showing the increase in visceral fat of different mice after consuming the high fat diet of stage three of Example 2;

FIG. 10 depicts a graph showing the liver triglyceride content of different mice after consuming the high fat diet of stage three of Example 2;

FIG. 11A depicts a graph showing the average rate of change in weight of the participants of Example 3;

FIG. 11B is a graph showing the average rate of change in abdominal circumference of the participants of Example 3;

FIG. 12A is a graph showing the average rate of change in visceral fat area of the participants of Example 3;

FIG. 12B is a comparison of CT scan images of the participants of Example 3;

FIG. 13 is a graph depicting the loss of visceral fat sustained by the participants of Example 4;

FIG. 14 is a graph depicting the visceral fat change rate sustained by the participants of Example 4;

FIG. 15 is a graph depicting the reduction in body weight sustained by the participants of Example 4;

FIG. 16 is a graph depicting the body weight change amount sustained by the participants of Example 4;

FIG. 17 is a graph depicting the reduction in BMI sustained by the participants of Example 4;

FIG. 18 is a graph depicting the BMI change amount sustained by the participants of Example 4;

FIG. 19 is a graph depicting the reduction in waist size sustained by the participants of Example 4;

FIG. 20 is a graph depicting the waist change amount sustained by the participants of Example 4;

FIG. 21 is a graph depicting the reduction in body fat ratio sustained by the participants of Example 4;

FIG. 22 is a graph depicting the body fat ratio change amount sustained by the participants of Example 4;

FIG. 23 is a graph depicting the reduction in subcutaneous fat area sustained by the participants of Example 4;

FIG. 24 is a graph depicting the subcutaneous fat area change rate sustained by the participants of Example 4;

FIG. 25 is a graph depicting the reduction in hip size that was sustained by the participants of Example 4; and

FIG. 26 is a graph depicting the hip change amount sustained by the participants of Example 4.

### DETAILED DESCRIPTION OF THE INVENTION

As used herein, the following terms and variations thereof have the meanings given below, unless a different meaning is clearly intended by the context in which such term is used.

Definitions of chemical terms and general terms used throughout the specification are described in more detail herein, but unless otherwise indicated the chemical elements are identified in accordance with the Periodic Table of the Elements, CAS version, Handbook of Chemistry and Physics, 75th Ed., inside cover, and specific functional groups if not specifically described herein are described by general principles of organic chemistry, as well as specific functional moieties and reactivity, as described in Organic Chemistry, 4th Edition, L. G. Wade, Jr., Prentice-Hall Inc., New Jersey, 1999.

The terms “a,” “an,” and “the” and similar referents used herein are to be construed to cover both the singular and the plural unless their usage in context indicates otherwise.

As used in this disclosure, the term “comprise” and variations of the term, such as “comprising” and “comprises,” are not intended to exclude other additives, components, integers, ingredients or steps.

“Isolation” or “purification” as used herein means separation of alkaloids or amino acids from other components in the alkaloid or amino acid starting material, which provides a substantially pure target compound, such as a substantially pure alkaloid. A compound or molecule which is “substantially pure” contains the compound or molecule in an amount of from about 50% to about 100%, from about 50% to about 80%, from about 70% to about 85%, from about 65% to about 95% by weight of the total compound or molecule in the material processed by the method of the invention.

The term “solution” refers to a composition comprising a solvent and a solute, and includes true solutions and suspensions. Examples of solutions include a solid, liquid or gas dissolved in a liquid and particulates or micelles suspended in a liquid.

The terms “individual,” “subject” “participant” and “patient” are used interchangeably herein, and generally refer to a mammal. The term “mammal” is defined as an individual belonging to the class Mammalia and includes, without limitation, humans, domestic and farm animals, and zoo, sports, and pet animals, such as cows, horses, sheep, dogs and cats.

The term “nutraceutical formulation” refers to a food or part of a food that offers medical and/or health benefits including prevention or treatment of disease. Nutraceutical products range from isolated nutrients, dietary supplements and diets, to genetically engineered designer foods, functional foods, herbal products and processed foods such as cereal, soup and beverages. The term “functional foods,” refers to foods that include “any modified food or food ingredients that may provide a health benefit beyond the traditional nutrients it contains.” Nutraceutical formulations of interest include foods for veterinary or human use, including food bars (e.g. cereal bars, breakfast bars, energy bars, nutritional bars); chewing gums; drinks; fortified drinks; drink supplements (e.g., powders to be added to a drink); tablets; lozenges; candies; and the like.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field. Preferred examples of such carriers or diluents include, but are not limited to, water, saline, Ringer's solutions, dextrose solution, PBS (phosphate-buffered saline), and 5% human serum albumin. Liposomes, cationic lipids and non-aqueous vehicles such as fixed oils may also be used. The use of such media and agents for pharmaceutically active substances is well known in the art. Except insofar as any conventional media or agent is incompatible with a therapeutic agent as defined above, use thereof in the composition of the present invention is contemplated.

A “therapeutic composition” as used herein means a substance that is intended to have a therapeutic effect such as pharmaceutical compositions, genetic materials, biologics, and other substances. Pharmaceutical compositions may be configured to function in inside the body with therapeutic qualities, concentration to reduce the frequency of replenishment, and the like.

As used herein, the phrases “therapeutically effective amount” and “prophylactically effective amount” refer to an amount that provides a therapeutic benefit in the treatment, prevention, or management of a disease or an overt symptom of the disease. The therapeutically effective amount may treat a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease. The specific amount that is therapeutically effective can be readily determined by ordinary medical practitioner, and may vary depending on factors known in the art, such as, e.g. the type of disease, the patient's history and age, the stage of disease, and the administration of other therapeutic agents.

The term “delivery system” refers to the formulation and delivery of the composition to a patient. Delivery systems include, but are not limited to, rapid dissolvable chewable or lozenges, liquid formulation, injection, compressed powder tablets, gel cap, transdermal gel or spray, intravenous delivery, time released liquid implants, electronic/substitute cigarettes or nasal sprays.

The present invention comprises methods for isolating a compound for use in weight loss and/or visceral fat reduction. In the present invention, the plant belonging to Umbelliferae refers to a plant belonging to Umbelliferae of Angiospermae, and the plant belonging to Umbelliferae is exemplified by, for example, Angelica keiskei koidzumi (or Ashitaba), Oenanthe javanica, Cryptotaenia japonica Hassk, Angelica pubescens, Daucus, Apium, Petroselium sativum and the like. In the present invention, Ashitaba is preferably utilized. In addition, although the entire plant belonging to Umbelliferae can be used, including the fruit, seed, seed coat, flower, leaf, stem, root, rhizome and/or whole plant, it is preferable that only the stem be used in the present invention.

Ashitaba comprises many active chemical compounds. These compounds comprise chalcones, coumarins, chroman, luteolin-7-glucoside, luteolin-7-O-α-D-glucpyranoside, 1-cerotol, daucosterol, stigmasterol, quercetin-3-O-β-D-glucopyranoside, steviol-I3-O-β-glucopyranoside 19-β-glucopyranosyl ester octaacetate, isoquercitrin, ruteorin, angelic acid, bergapten, beta-carotene, Vitamin C, Vitamin B12, Vitamin B2, Vitamin K, potassium, calcium, iron and chlorophyll.

The chalcones compounds can comprise one or more of the following sub-compounds: Ashitaba-chalcone, xanthoangelol, xanthoangelol-B, xanthoangelol-C, xanthoangelol-D, xanthoangelol-E, xanthoangelol-F, xanthoangelol-G, xanthoangelol-H, xanthoangelol-I, xanthoangelol-J, isobavachalcone, deoxydihydroxanthoangelol-H, 4-hydroxyderricin, xanthohumol, and xanthokeismin A.

The coumarin compounds can comprise one or more of the following sub-compounds: psoralen, imperatorin, columbianagin, isorhazelpitin, rhazelpiton, selenidin, xanthotoxin, angelicin, archangelicin, and 8(S), 9(S)-angeloyloxyl-8,9-dihydrooroselol.

The weight loss/visceral fat reduction compound of the present invention comprises at least 8.0% total chalcones. The total chalcones can comprise 4-hydroxyderricin and xanthoangelol. The at least 8% total chalcones can comprise any percentage of 4-hydroxyderricin and xanthoangelol, but preferably at least 3% of the at least 8% total chalcones comprises 4-hydroxyderricin and at least 5% of the at least 8% total chalcones comprises xanthoangelol.

Preferably, the coumarins comprising the compound of the present invention comprise furanocoumarin type coumarins and dihydropyrano-coumarins. Preferably the furanocoumarins are selected from the group comprising xanthotoxin (psoralen), isopimpinellin, bergapten, and imperatorin. Preferably the dihydropyrano-coumarins are selected from a group comprising laserpitin, isolaserpitin, and selinidin.

Table 1 provides example concentrations and total coumarin content that can comprise the present invention.

TABLE 1
Example Concentrations and Total Coumarin Content

| | | | Conc. | Total coumarins: | Total coumarins |
|---|---|---|---|---|---|
| Lot | Type | Components | (mg/g) | (mg/g) | (mg/g) |
| IACC0910 | Liner-type | Xanthotoxin (Psoralen) | 0.711 | 1.70 | 36.19 |
| | furanocoumarins | Isopimpinellin | 0.782 | | |
| | | Bergapten | 0.205 | | |
| | | Imperatorin | <0.001 | | |
| | Angular-type | Laserpitin | 13.599 | 34.49 | |
| | dihydropyrano- | Isolaserpitin | 15.193 | | |
| | coumarins | Selinidin | 5.694 | | |
| IACD0707 | Liner-type | Xanthotoxin (Psoralen) | 0.795 | 1.99 | 35.11 |
| | furanocoumarins | Isopimpinellin | 0.937 | | |
| | | Bergapten | 0.260 | | |
| | | Imperatorin | <0.001 | | |
| | Angular-type | Laserpitin | 13.059 | 33.12 | |
| | dihydropyrano- | Isolaserpitin | 14.404 | | |
| | coumarins | Selinidin | 5.655 | | |
| IACE0322 | Liner-type | Xanthotoxin (Psoralen) | 0.774 | 1.91 | 44.55 |
| | furanocoumarins | Isopimpinellin | 0.928 | | |
| | | Bergapten | 0.211 | | |
| | | Imperatorin | <0.001 | | |
| | Angular-type | Laserpitin | 15.560 | 42.64 | |
| | dihydropyrano- | Isolaserpitin | 19.534 | | |
| | coumarins | Selinidin | 7.544 | | |
| Average | Liner-type | Xanthotoxin (Psoralen) | 0.760 | 1.87 | 38.02 |
| | furanocoumarins | Isopimpinellin | 0.882 | | |
| | | Bergapten | 0.225 | | |
| | | Imperatorin | <0.001 | | |
| | Angular-type | Laserpitin | 14.070 | 36.75 | |
| | dihydropyrano- | Isolaserpitin | 16.377 | | |
| | coumarins | Selinidin | 6.298 | | |

The typical method of extracting chalcones and coumarins from Ashitaba comprises a hot-air dried powder formed from the pulverized leaves and stems of the Ashitaba plant. The color of the powder is typically bright green. As the leaves of the Ashitaba plant typically contain approximately 0.25% to 0.35% chalcones, the resulting powder comprises a relatively low concentration of chalcones.

The method of isolation and extraction of the present invention is specifically directed to the sap that exudes from the stem of the Ashitaba plant. Ashitaba contains a thick, sticky yellow sap which is unique to this strain of Angelica.

The isolation and extraction procedure of chalcones and coumarins in the present invention comprises first cutting a stem of an Ashitaba plant, so that sap begins to exude from the cut stem. Next, the sap is harvested from the cut stem. Once the sap has been harvested, it is pasteurized at 90° C. for 30 minutes. Next, the pasteurized sap is mixed with cyclodextrin to produce a mixture. Then, the mixture is sterilized by heating for 15 minutes at 121° C. Once the mixture has been sterilized, the mixture is then freeze-dried. Once the mixture is freeze-dried, it is then passed through a 100 mesh to pulverize the mixture. Once the mixture has been pulverized, it results in a purified powder containing chalcones and coumarins. The powder can be consumed as a loose powder which can be mixed into a food or drink, or the loose powder can be formed into capsules for ease of consumption.

### EXAMPLES

### Example 1

### Method of Purification of Ashitaba Chalcone Powder

In this example, a method of purification of Ashitaba chalcone powder was studied.

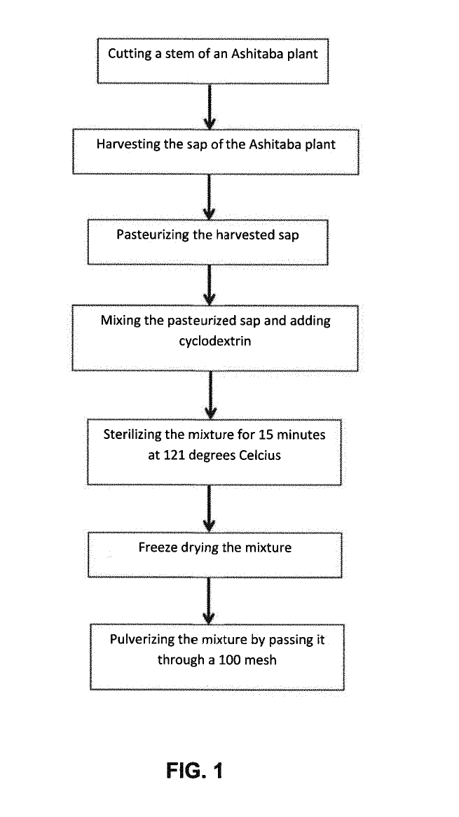

The method of purification of Ashitaba chalcone powder comprises the steps of: first cutting a stem of an Ashitaba plant, so that sap begins to exude from the cut stem. Next, the sap is harvested from the cut stem. Once the sap has been harvested, it is pasteurized at 90° C. for 30 minutes. Next, the pasteurized sap is mixed with cyclodextrin to produce a mixture. Then, the mixture is sterilized by heating for 15 minutes at 121° C. Once the mixture has been sterilized, the mixture is then freeze-dried. Once the mixture is freeze-dried, it is then passed through a 100 mesh to pulverize the mixture. Once the mixture has been pulverized, it results in a purified powder containing chalcones and coumarins. The powder can be consumed as a loose powder which can be mixed into a food or drink, or the loose powder can be formed into capsules for ease of consumption. FIG. 1 depicts a flow chart demonstrating the method of purification.

### Example 2

### Inhibitory Effects of Ashitaba Chalcone on Fat Accumulation in Mice

In this example, the inhibitory effects of Ashitaba chalcones on fat accumulation in mice was researched.

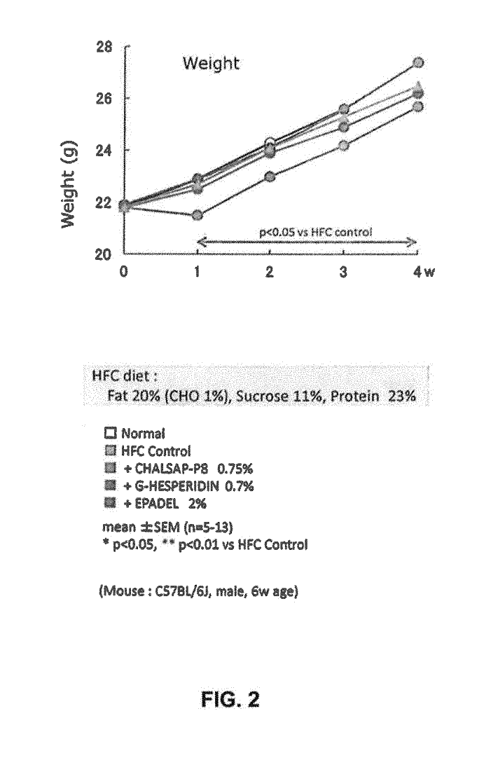

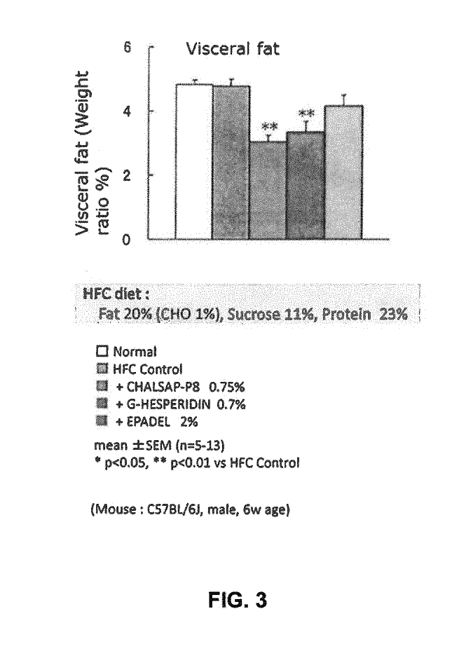

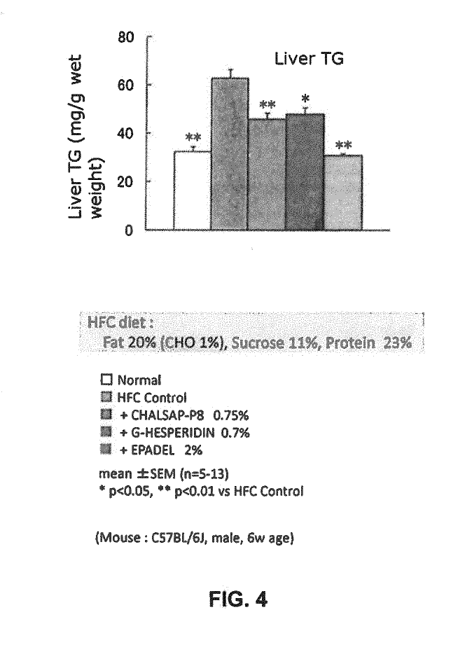

In the first stage of the study, the mice were given a high fat and high cholesterol food comprising 20% fat, 1% cholesterol, 11% sucrose and 23% protein for 4 weeks. The mice that received the Ashitaba chalcone compound gained less weight, gained less visceral fat and the liver triglyceride content also increased less than the other mice that did not receive the Ashitaba chalcone compound. FIG. 2 depicts a graph showing the weight gain of the different mice. FIG. 3 depicts a graph showing the increase in visceral fat of the different mice, and FIG. 4 depicts a graph showing the liver triglyceride content of the different mice.

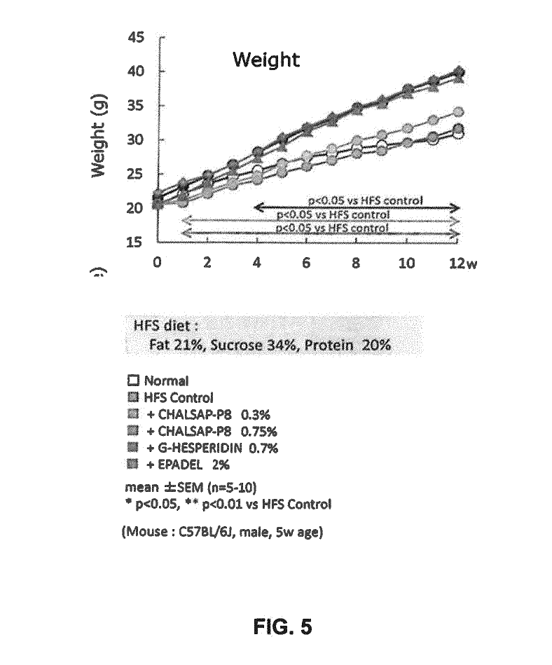

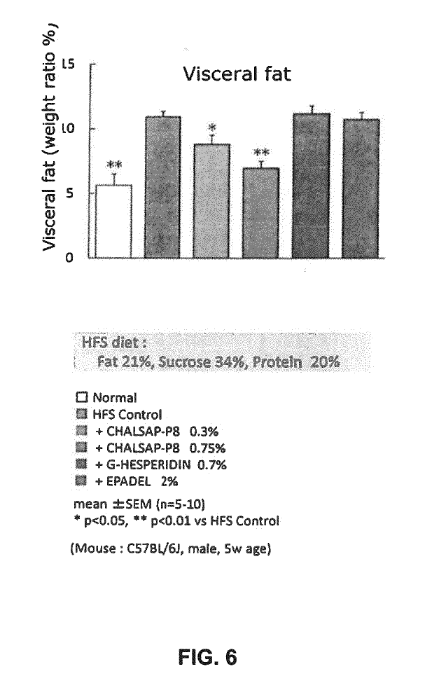

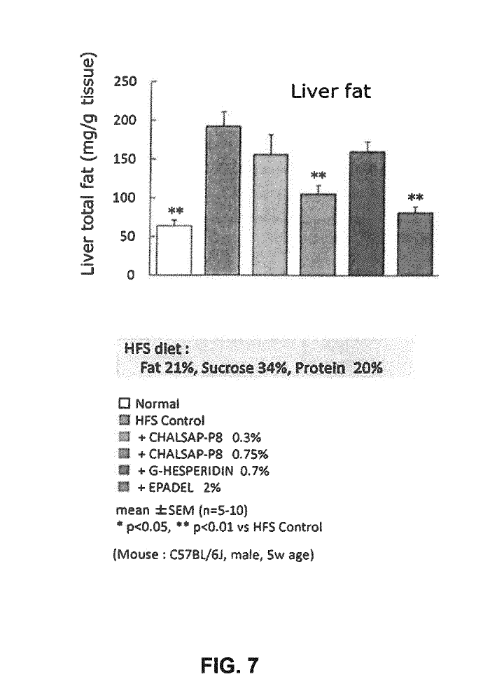

In the second stage of this study, the mice were given a high fat high sugar food comprising 21% fat, 34% sucrose and 20% protein for 12 weeks. The mice that received the Ashitaba chalcone compound gained less weight, gained less visceral fat and their total liver fat was less than the mice that did not receive the Ashitaba chalcone compound. FIG. 5 depicts a graph showing the weight gain of the different mice, FIG. 6 depicts a graph showing the increase in visceral fat of the different mice and FIG. 7 depicts a graph showing the total liver fat of the different mice.

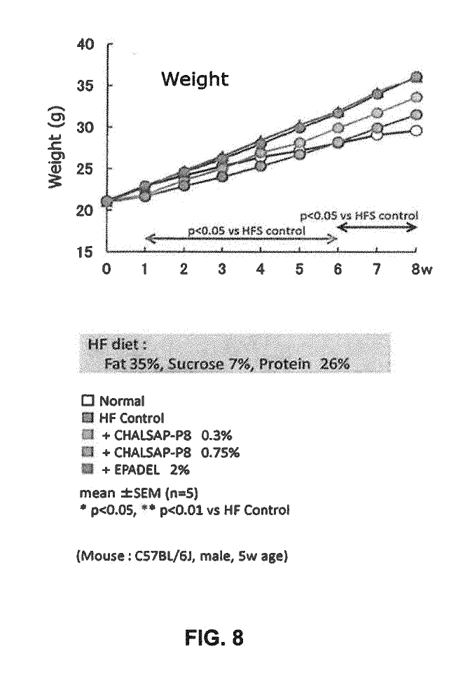

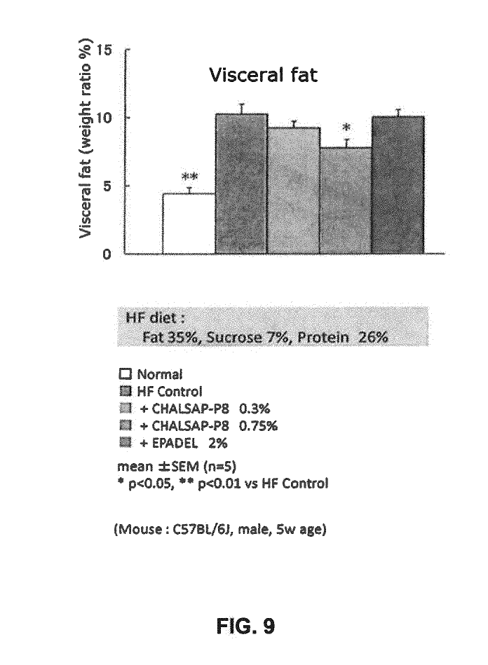

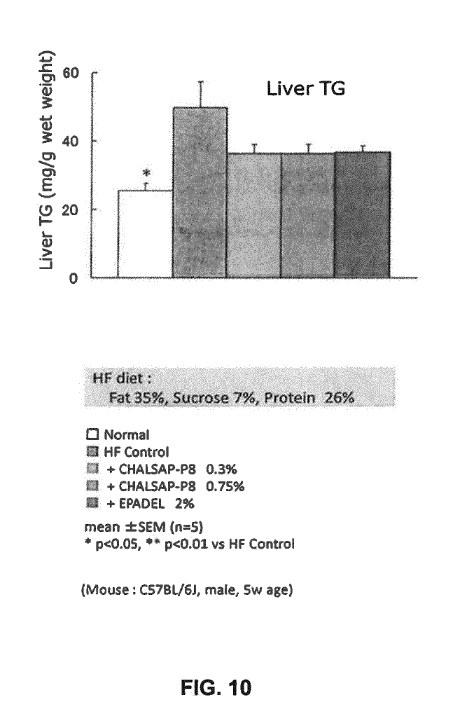

In the third stage of this study, the mice were given a high fat food comprising 35% fat, 7% sucrose, and 26% protein for 8 weeks. The mice that received the Ashitaba chalcone compound gained less weight, gained less visceral fat and their total liver fat was less than the mice that did not receive the Ashitaba chalcone compound. FIG. 8 depicts a graph showing the weight gain of the different mice, FIG. 9 depicts a graph showing the increase in visceral fat of the different mice and FIG. 10 depicts a graph showing the total liver fat of the different mice.

Table 2 depicts a summary of the study results.

### Example 3

### Anti-Metabolic Syndrome Action of Ashitaba Chalcone Powder

In this example, the anti-metabolic syndrome action by administration of Ashitaba chalcone powder containing a total chalcone content of more than 8% was verified.

This was a randomized, placebo-controlled, double-blind study. One group was administered the Ashitaba chalcone powder (containing a total chalcone content of more than 8%), and the other group was administered the placebo after dinner every day for 8 weeks. The participants' blood was collected and their abdominal circumference and weight were measured before the start of the test and in Week 2, 4 and 8. The participants' visceral fat area was also measured by CT scanning before the start of the test and in Week 8. The participants comprised 15 healthy males (Ave. age 38, Ave. BMI 24.0 ±3.2). 10 males received the test food, and 5 males received the placebo.

The test food comprised hard shell capsules containing 200 mg of Ashitaba chalcone powder which were taken once a day. The placebo comprised hard shell capsules containing indigestible dextrin (200 mg/day)

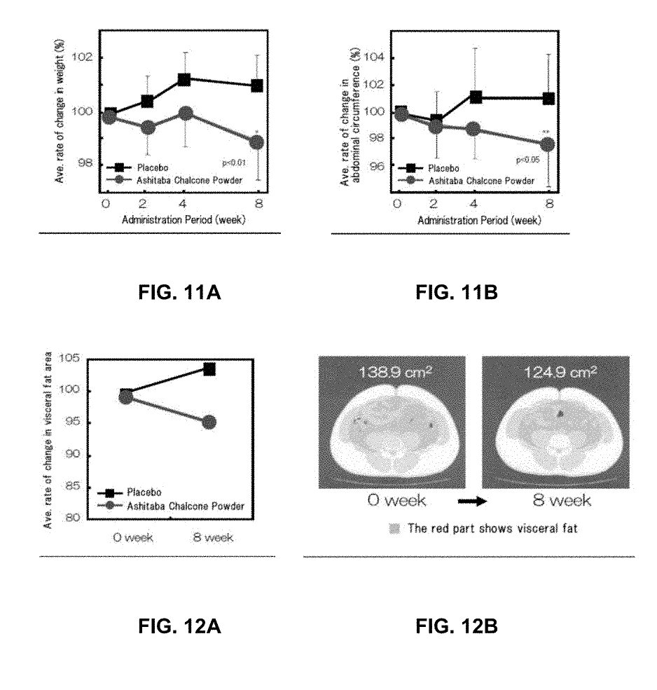

The average rate of changes in the participants' abdominal circumference and weight are shown in FIGS. 11A and 11B. There was a significant decrease for both abdominal circumference and weight in the testing group. The average rate of changes in the participants' visceral fat is shown in FIG. 12A. There was a tendency to decrease in the visceral fat in the testing group. The decrease in visceral fat was compared also by images of CT scan, shown in FIG. 12B.

It was confirmed there was a significant decrease in abdominal circumference and weight by administration of Ashitaba chalcone powder. It was found that these decreases were due to the decrease in the visceral fat. The participants with a larger amount of visceral fat noticed a higher rate of decrease; however no change was found in those who were at the normal range of visceral fat amount and their visceral fat did not decrease too much. The participants did not have any abnormal biochemical levels in the blood and any influence in their health.

### Example 4

### Reduction of Body Fat From Consuming Ashitaba Chalcone

In this example, the reduction of body fat when food containing Ashitaba chalcone powder was consumed was tested. It was a double-blind, placebo-controlled study on slightly obese male and female adults with BMI equal to or more than 25 and less than 30.

The subjects, after dinner, took one capsule of either a test food containing 200 mg of Ashitaba chalcone or a placebo food without Ashitaba chalcone for 56 days.

The participants weight, BMI, body fat area (which comprises visceral fat, subcutaneous fat and the total fat), waist circumference, hip circumference, clinical test data, special test (PAI-1 (plasminogen activator inhibitor-1) antigen, tPA (tissue plasminogen activator) activity, ECLT (euglobulin clot lysis time), adiponectin, resistin, leptin), blood pressure, heart rate and diet were all evaluated and recorded.

26 participants were randomly assigned to either the placebo group (male: 9, female: 4) or the test group (male: 9, female 4). The mean±standard deviation of extraneous factors of the participants is shown in Table 3. In the placebo group, these were age 52.4±6.9 years old, height 165.00±8.94 cm, body weight 72.47±7.11 kg, and BMI 26.59±1.08. In the treatment group, these were age 47.3±5.9 years old, height 165.60±6.57 cm, body weight 73.85±6.36 kg, and BMI 26.92±1.41.

TABLE 3
Mean ± Standard Deviation of Extraneous Factors of the Participants

| Item | Unit | Sex | Food | Number | Pre-test |
|---|---|---|---|---|---|
| Age | years-old | All | P | 13 | 52.4 ± 6.9 |
| | | | A | 13 | 47.3 ± 5.9 |
| | | Male | P | 9 | 53.7 ± 6.7 |
| | | | A | 9 | 46.6 ± 5.6 |
| | | Female | P | 4 | 49.5 ± 7.4 |
| | | | A | 4 | 49.0 ± 7.0 |
| Height | (cm) | All | P | 13 | 165.00 ± 8.94 |
| | | | A | 13 | 165.60 ± 6.57 |
| | | Male | P | 9 | 169.32 ± 5.29 |
| | | | A | 9 | 167.63 ± 6.59 |
| | | Female | P | 4 | 155.28 ± 7.95 |
| | | | A | 4 | 161.03 ± 4.10 |
| Weight | (kg) | All | P | 13 | 72.47 ± 7.11 |
| | | | A | 13 | 73.85 ± 6.36 |
| | | Male | P | 9 | 75.80 ± 5.42 |
| | | | A | 9 | 76.44 ± 5.79 |
| | | Female | P | 4 | 64.98 ± 3.92 |
| | | | A | 4 | 68.03 ± 2.67 |
| BMI | | All | P | 13 | 26.59 ± 1.08 |
| | | | A | 13 | 26.92 ± 1.41 |
| | | Male | P | 9 | 26.41 ± 0.79 |
| | | | A | 9 | 27.21 ± 1.34 |
| | | Female | P | 4 | 27.00 ± 1.64 |
| | | | A | 4 | 26.25 ± 1.51 |
| Body fat ratio | (%) | All | P | 13 | 28.48 ± 4.84 |
| | | | A | 13 | 29.13 ± 4.37 |
| | | Male | P | 9 | 25.89 ± 2.64 |
| | | | A | 9 | 26.99 ± 3.12 |
| | | Female | P | 4 | 34.30 ± 3.11 |
| | | | A | 4 | 33.95 ± 2.38 |

Value is the mean ± standard deviation.
P: Placebo,
A: Chalcone

One capsule (200 mg) per day was taken with a glass of water after dinner. When forgotten, it was taken within the same day. The intake period was 56 days (i.e., 8 weeks). The principal ingredient was Ashitaba chalcone.

TABLE 4
Composition of the Test Food per 1 Capsule (200 mg)

| | |
|---|---|
| Form | Two-piece capsule |
| Ingredients | 98% Ashitaba Chalcone Powder (made by Japan Bio |
| | Science Laboratory Co., Ltd.) |
| | 2% Ester (Product name: DK Ester F-20W, made by |
| | Dai-ichi Kogyo Seiyaku Co., Ltd.) |
| Amount of the | 196 mg as Ashitaba chalcone (Chalcone content: |
| principal ingredient | about 16 mg) |
| Shelf life | 24 months |
| Storage | At room temperature (Measured values during the |
| | storage period: 19 to 29 degrees C.) |

*Chalcone is an active ingredient contained in Ashitaba and mainly means xanthoangelol and 4-hydroxyderricin.

TABLE 5
Composition of the Placebo Food per 1 capsule (200 mg)

| | |
|---|---|
| Form | Two-piece capsule |
| Ingredients | 98% Cyclodextrin (Product name: Isoelite-P, made |
| | by Kansai Seito K. K. |
| | 2% Ester (Product name: DK Ester F-20W, made by |
| | Dai-ichi Kogyo Seiyaku Co., Ltd.) |
| Amount of principal | 0 mg as Ashitaba chalcone |
| ingredient | |
| Shelf life | 24 months |
| Storage | At room temperature |

The participants were randomly assigned to two groups (each group consist of 13 participants). Each participant received assigned foods and started the intake from the determined date. The participants came to the hospital at the 4th and 8th week after the intake, where several tests (e.g., measurement of the body fat) were performed.

The evaluation method consisted of performing a between-groups (treatment group vs. placebo group) t-test on the change amount 8 weeks after the intake. It also evaluated the change amount 4 weeks after the intake as a reference. In addition, a one-sample t-test on the change amount at each time period from the baseline was performed. Any additional analysis was also performed as needed.

There was no significant difference between the placebo group and the treatment group in the primary end points, namely, the visceral fat area and body weight, because these values decreased in both groups. However, only in the treatment group, compared to the baseline (i.e., before the intake of the test food), were a significant reduction of the visceral fat area at 8th week and body weight at 4th and 8th week confirmed.

Like body weight, the secondary end point, BMI, did not differ significantly between the groups. However, a significant decrease was observed only in the treatment group when the value before the intake was compared to that of 4th and 8th week. Although there was no significant difference in the value of waist circumstance between the groups, each group showed the significant reduction at 4th and 8th week compared to that of the baseline. The body fat percentage of both group decreased at 4th and 8th week after the intake. However, the significant reduction was confirmed only in the placebo group at 8th week after the intake.

Regarding the other end points, the total fat area decreased in the both groups at 8th week after the intake, but a significant reduction was confirmed only in the treatment group. The hip circumstance significantly decreased in the placebo group at 8th week and in the treatment group at 4th and 8th week after the intake.

The significant reduction of the visceral fat area and body weight were confirmed within the treatment group although the difference between the groups was not confirmed. Therefore, the reduction effect on the visceral fat area and the body weight by the intake of Ashitaba chalcone were what we could expect.

The average intake rate of the placebo group was 99.1% (100%=11, 95%=1, 93%=1). The average intake rate of the treatment group was 98.2% (100%=8, 98%=1, 96%=2, 93%=2)

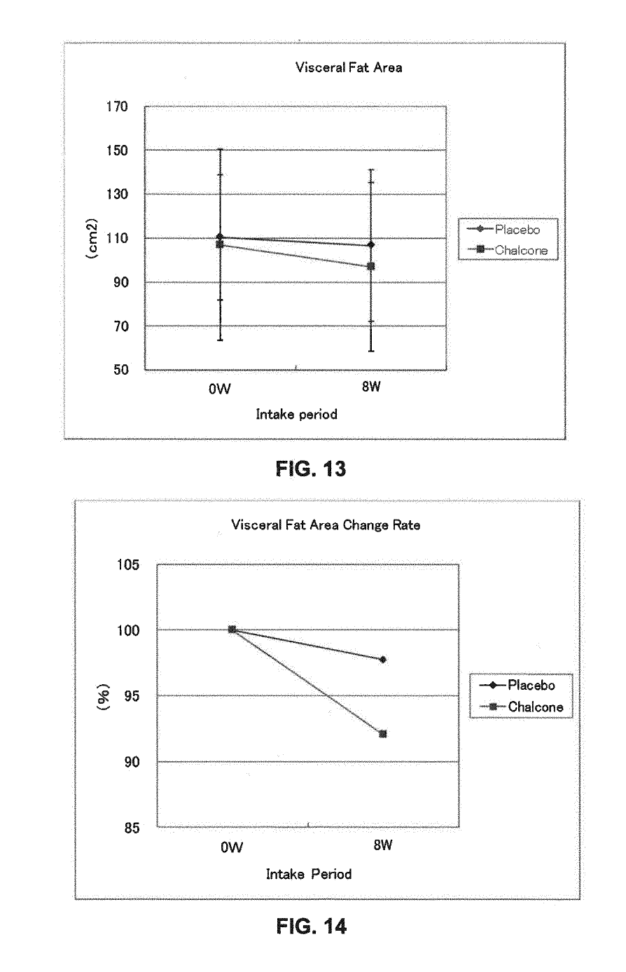

The mean±standard deviation, the amount of change and the rate of change with respect to body fat area are shown in Table 6, and their transition is shown in FIGS. 13-14.

TABLE 6
Body Fat Area

| | | | | | | | Change | Change rate |
|---|---|---|---|---|---|---|---|---|
| Item | Unit | Sex | Food | Number | Baseline | 8th week | amount | (%) |
| Visceral fat | (cm²) | All | P | 13 | 110.35 ± 28.63 | 106.71 ± 34.52 | −3.64 ± 26.43 | 97.73 ± 25.63 |
| area | | | A | 13 | 107.00 ± 43.56 | 96.98 ± 38.41* | −10.02 ± 15.91 | 92.06 ± 14.55 |
| | | Male | P | 9 | 120.86 ± 27.43 | 109.50 ± 33.55 | −11.36 ± 20.26 | 90.53 ± 16.69 |
| | | | A | 4 | 123.30 ± 43.27 | 107.32 ± 42.31* | −15.98 ± 15.24 | 86.49 ± 13.06 |
| | | Female | P | 9 | 86.70 ± 13.96 | 100.43 ± 41.10 | 13.73 ± 33.46 | 113.94 ± 37.12 |
| | | | A | 4 | 70.33 ± 2.60 | 73.73 ± 9.27 | 3.40 ± 6.80 | 104.59 ± 9.48 |
| Subcutaneous | (cm²) | All | P | 13 | 184.94 ± 51.94 | 180.33 ± 51.41 | −4.61 ± 22.10 | 98.87 ± 13.12 |
| fat area | | | A | 13 | 223.27 ± 56.69 | 214.13 ± 55.33 | −9.14 ± 21.88 | 96.79 ± 10.18 |
| | | Male | P | 9 | 165.53 ± 42.21 | 164.94 ± 35.93 | −0.59 ± 17.48 | 101.54 ± 12.97 |
| | | | A | 4 | 208.29 ± 54.64 | 203.38 ± 46.86 | −4.91 ± 18.79 | 99.06 ± 9.44 |
| | | Female | P | 9 | 228.60 ± 48.66 | 214.95 ± 69.44 | −13.65 ± 31.32 | 92.84 ± 13.06 |
| | | | A | 4 | 256.98 ± 51.99 | 238.33 ± 72.57 | −18.65 ± 28.28 | 91.70 ± 11.26 |
| Total fat | (cm²) | All | P | 13 | 295.28 ± 54.16 | 287.04 ± 63.40 | −8.25 ± 29.91 | 97.11 ± 10.05 |
| area | | | A | 13 | 330.27 ± 59.57 | 311.12 ± 66.26* | −19.15 ± 27.19 | 94.01 ± 8.42 |
| | | Male | P | 9 | 286.39 ± 56.96 | 274.44 ± 58.33 | −11.94 ± 27.39 | 96.07 ± 9.85 |
| | | | A | 4 | 331.59 ± 65.83 | 310.70 ± 68.49* | −20.89 ± 27.15 | 93.64 ± 8.32 |
| | | Female | P | 9 | 315.30 ± 48.10 | 315.38 ± 73.87 | 0.07 ± 37.99 | 99.43 ± 11.62 |
| | | | A | 4 | 327.30 ± 51.21 | 312.05 ± 71.09 | −15.25 ± 31.01 | 94.83 ± 9.87 |

Value is the mean ± standard deviation.
P: Placebo,
A: Chalcone
*p < 0.05,
**: p < 0.01 (One sample t-test comparing baseline),
#: p < 0.05,
##: p < 0.01 (two sample t-test comparing placebo group and chalcone group)

The visceral fat area decreased 8 weeks after the intake in both groups, but the difference between the two groups was not significant. However, the amount of the reduction (i.e., the change amount and the change rate) was large and significant compared to that of the baseline (the baseline→8th week: 107.00±43.56→96.98±38.41 cm2, a reduction of 7.94%). On the other hand, the reduction was not significant in the placebo group. There was no significant difference in the change amount and the change rate. In addition, when the subgroup analysis of male and female was performed, there was a significant reduction in the male participants of the treatment group at 8th week compared to the baseline (the baseline→8th week: 123.3±43.27→107.32±42.31 cm2, a reduction of 13.51%).

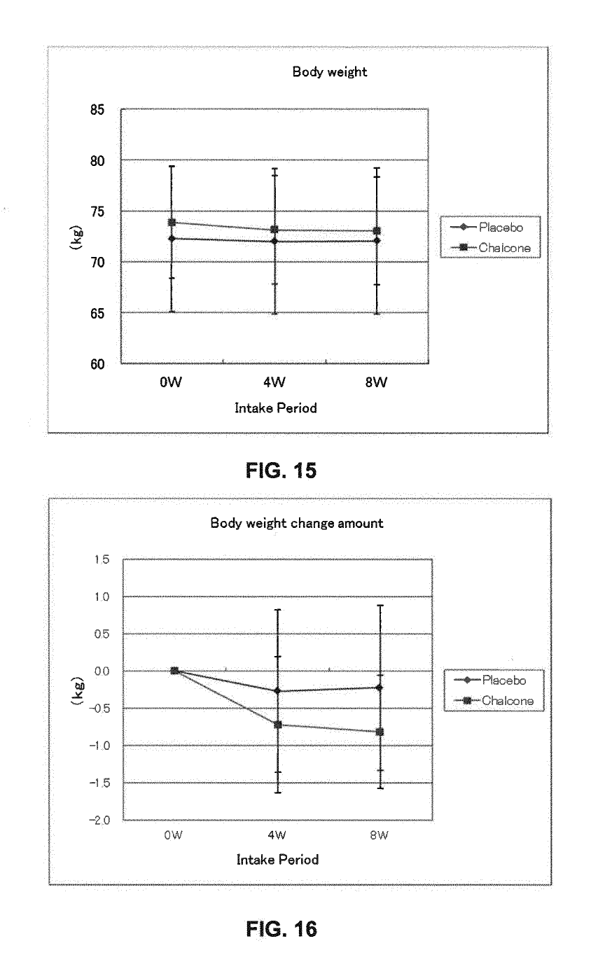

Table 7 shows the mean±standard deviation of the body weight and the Tables 9 and 10 show the change amount and the change rate. The transition is shown in FIGS. 15-16.

TABLE 7
Body Measurements (Body Weight, BMI, Body Fat Ratio)

| Item | Unit | Sex | Food | Number | Baseline | 4th week | 8th week |
|---|---|---|---|---|---|---|---|
| Body | (kg) | All | P | 13 | 72.28 ± 7.13 | 72.01 ± 7.15 | 72.05 ± 7.16 |
| weight | | | A | 13 | 73.86 ± 6.15 | 73.14 ± 5.98* | 73.05 ± 6.21** |
| | | Male | P | 9 | 75.66 ± 5.23 | 75.46 ± 5.15 | 75.44 ± 5.27 |
| | | | A | 9 | 76.49 ± 5.49 | 75.67 ± 5.32* | 75.79 ± 5.29* |
| | | Female | P | 4 | 64.68 ± 4.40 | 64.25 ± 4.23 | 64.43 ± 4.35 |
| | | | A | 4 | 67.95 ± 1.92 | 67.45 ± 2.23 | 66.88 ± 2.50 |
| BMI | | All | P | 13 | 26.52 ± 1.01 | 26.41 ± 1.17 | 26.44 ± 1.21 |
| | | | A | 13 | 26.92 ± 1.36 | 26.67 ± 1.41* | 26.64 ± 1.45** |
| | | Male | P | 9 | 26.37 ± 0.83 | 26.29 ± 0.76 | 26.28 ± 0.76 |
| | | | A | 9 | 27.21 ± 1.37 | 26.94 ± 1.43 | 27.00 ± 1.49* |
| | | Female | P | 4 | 26.85 ± 1.42 | 26.68 ± 1.95 | 26.80 ± 2.02 |
| | | | A | 4 | 26.25 ± 1.22 | 26.05 ± 1.31 | 25.83 ± 1.06 |
| Body | (%) | All | P | 13 | 28.07 ± 4.89 | 27.58 ± 5.04 | 27.35 ± 4.85** |
| fat ratio | | | A | 13 | 28.73 ± 4.38 | 28.31 ± 4.13 | 27.95 ± 4.43 |
| | | Male | P | 9 | 25.50 ± 2.51 | 24.81 ± 2.34 | 24.87 ± 2.25** |
| | | | A | 9 | 26.61 ± 3.05 | 26.32 ± 3.12 | 25.68 ± 3.12 |
| | | Female | P | 4 | 33.85 ± 3.82 | 33.83 ± 3.47 | 32.93 ± 4.55 |
| | | | A | 4 | 33.50 ± 2.84 | 32.78 ± 1.96 | 33.08 ± 1.47 |

Value is the mean ± standard deviation.
P: Placebo,
A: Chalcone
*p < 0.05,
**p < 0.01 (One sample t-test comparing baseline),
#: p < 0.05,
##: p < 0.01 (two sample t-test comparing placebo group and chalcone group)

There was a reduction at 4th week and 8the week in both groups, but it was significant only in the treatment group (the baseline→4th week→8th week: 73.86±6.15→73.14±5.98→73.05±6.21 kg, a reduction of 0.81 kg at 8th week). There was no significant group difference in the change amount and in the change rate.

When the subgroup analysis of male and female was conducted, the male participants of the treatment group showed a significant reduction at 4th and 8th week (the baseline→4th week→8th week: 76.49±5.49→75.67±5.32→75.79±5.29 kg, a reduction of 0.70 kg at 8th week). The reduction in the female participants of the treatment group was not significant but it was −1.07 kg at 8th week and larger than that of the male participants.

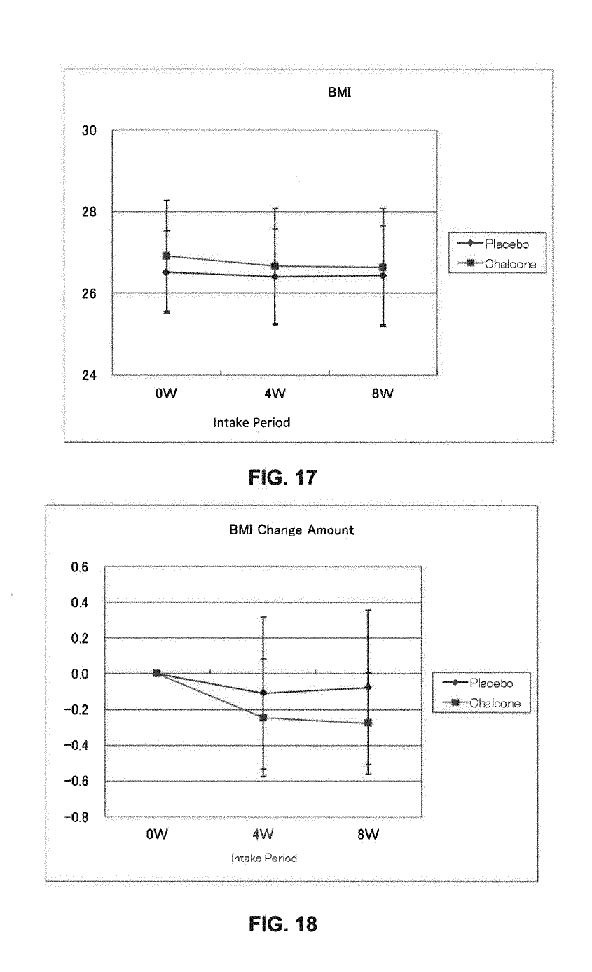

The mean±standard deviation of BMI is shown in Table 7. The change amount and the change rate are shown in FIGS. 17-18. The BMI, like the body weight, decreased in both groups, but the reduction was significant at 4th and 8th week only in the treatment group (the baseline→4th week→8th week: 26.92±1.36→26.67±1.41→26.64±1.45, a reduction of 0.28 at 8th week). When the subgroup analysis of male and female was performed, there was a significant reduction in the male participants of the treatment group at 8th week (the baseline→8th week: 27.21±1.37→27.00±1.49, a reduction of 0.21 at 8th week). Like the body weight, the reduction was not significant in the female of the treatment group but it was −0.42 at 8th week and a larger than that of the male participants.

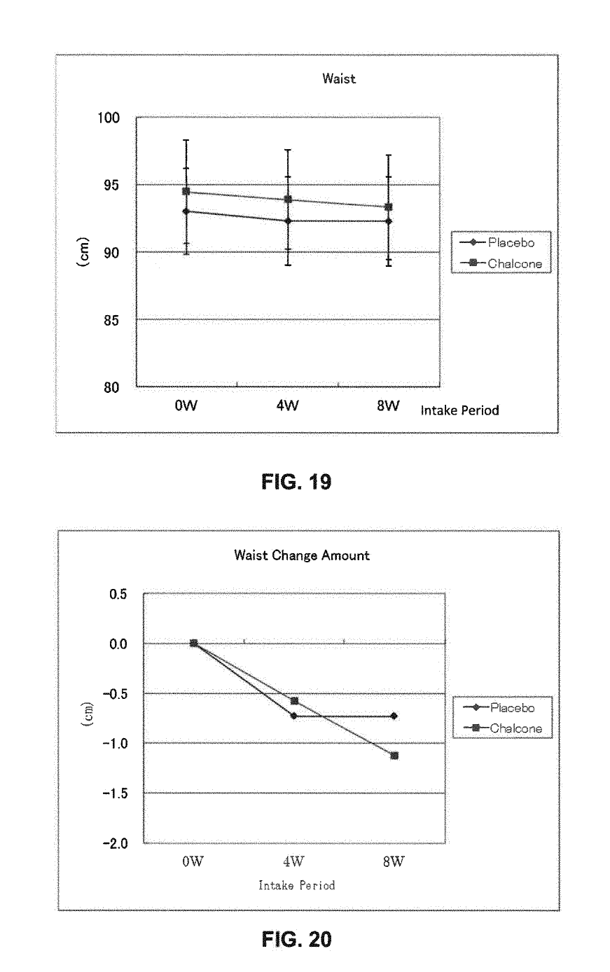

The mean±standard deviation of the waist circumference is shown in Table 8. The change amount and the change rate are shown in FIGS. 19-20.

TABLE 8
Body Measurements (Waist, Hip, Body Fat Ratio)

| Item | Unit | Sex | Food | Number | Pre-Inspection | Baseline | 4th week | 8th week |
|---|---|---|---|---|---|---|---|---|
| Waist | (cm) | All | P | 13 | 93.14 ± 3.23 | 93.02 ± 3.20 | 92.29 ± 3.29** | 92.29 ± 3.29* |
| | | | A | 13 | 94.68 ± 3.95 | 94.48 ± 3.84 | 93.90 ± 3.67** | 93.35 ± 3.88** |
| | | Male | P | 9 | 93.72 ± 3.79 | 93.58 ± 3.76 | 92.82 ± 3.82** | 92.80 ± 3.83** |
| | | | A | 9 | 95.07 ± 3.51 | 94.79 ± 3.45 | 94.13 ± 3.17** | 93.71 ± 3.21** |
| | | Female | P | 4 | 91.83 ± 0.43 | 91.78 ± 0.29 | 91.10 ± 1.21 | 91.15 ± 1.32 |
| | | | A | 4 | 93.80 ± 5.30 | 93.78 ± 5.13 | 93.38 ± 5.16* | 92.55 ± 5.62 |
| Hip | (cm) | All | P | 13 | 98.85 ± 3.23 | 98.48 ± 3.22 | 98.25 ± 3.09 | 98.02 ± 3.14* |
| | | | A | 13 | 100.38 ± 3.25 | 100.07 ± 3.29 | 99.61 ± 3.23** | 99.38 ± 3.20** |
| | | Male | P | 9 | 100.12 ± 2.81 | 99.72 ± 2.81 | 99.42 ± 2.74 | 99.18 ± 2.91* |
| | | | A | 9 | 99.74 ± 3.56 | 99.47 ± 3.65 | 99.01 ± 3.60* | 98.97 ± 3.64** |
| | | Female | P | 4 | 95.98 ± 2.19## | 95.68 ± 2.32## | 95.63 ± 2.22# | 95.43 ± 1.95# |
| | | | A | 4 | 101.83 ± 2.11 | 101.43 ± 2.03 | 100.95 ± 1.93 | 100.33 ± 1.96 |
| W/H | | All | P | 13 | 0.943 ± 0.032 | 0.945 ± 0.032 | 0.940 ± 0.030* | 0.942 ± 0.031 |
| | | | A | 13 | 0.944 ± 0.038 | 0.945 ± 0.036 | 0.943 ± 0.037 | 0.940 ± 0.038* |
| | | Male | P | 9 | 0.936 ± 0.034 | 0.939 ± 0.034 | 0.934 ± 0.031* | 0.936 ± 0.033 |
| | | | A | 9 | 0.954 ± 0.033 | 0.953 ± 0.031 | 0.951 ± 0.032 | 0.947 ± 0.031* |
| | | Female | P | 4 | 0.957 ± 0.024 | 0.960 ± 0.026 | 0.953 ± 0.027 | 0.956 ± 0.025 |
| | | | A | 4 | 0.921 ± 0.044 | 0.924 ± 0.044 | 0.925 ± 0.047 | 0.922 ± 0.050 |

Value is the mean ± standard deviation.
P: Placebo,
A: Chalcone
*p < 0.05,
**p < 0.01 (One sample t-test comparing baseline),
#p < 0.05,
##p < 0.01 (two sample t-test comparing placebo group and chalcone group)

There was a significant reduction at 4th and 8th week in both groups. The significant group difference was not observed but the decrease of the treatment group at 8th week (−1.13 cm) was greater than that of the placebo group (−0.73 cm).

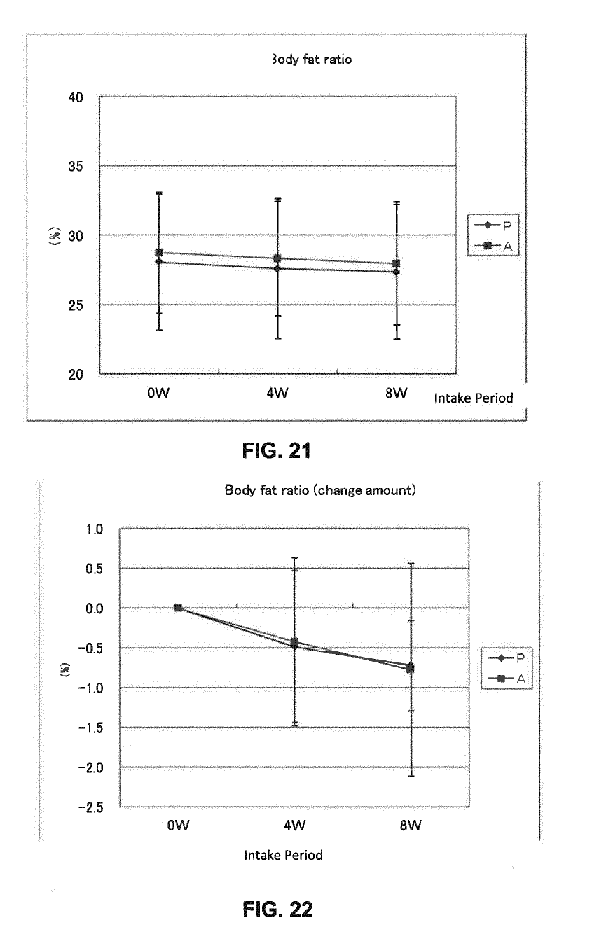

The mean±standard deviation of the body fat ratio is shown in Table 7. The change amount and the change rate are shown in FIGS. 21-22. The body fat ratio decreased at 4th and 8th week in both groups, but it was significant only in the placebo group at 8the week.

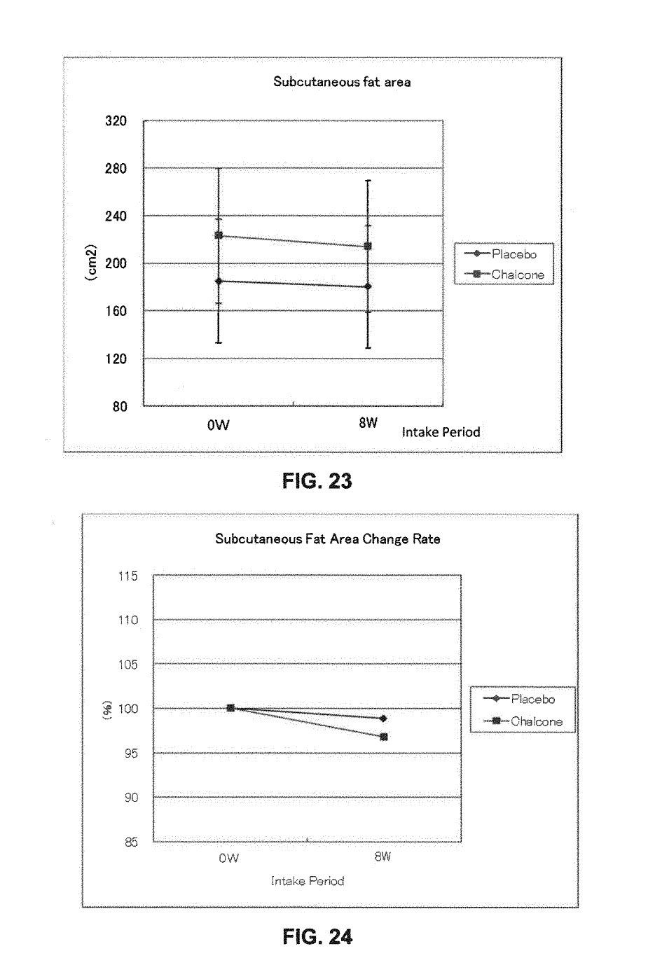

Table 6 shows the mean±standard deviation of the subcutaneous fat area and the total fat area, the change amount and the change rate. The transition is shown in FIGS. 23-24. The subcutaneous fat area decreased at 8th week in both groups, but it was not significant. The difference between groups was not significant, but the reduction of the placebo group at 8 the week was 1.13% while that of the treatment group was 3.21%. The male participants of both groups did not show a reduction, but the female participants of the placebo group and the treatment group had a reduction of 7.16% and 8.30%, respectively. This suggested that the rate of the decrease was greater in the female participants.

In the total fat area, both group showed a reduction at 8th week, but it was significant only in the treatment group compared to the baseline (baseline→8th week: 330.27±59.57→311.12±66.26 cm², a reduction of 5.99%).

In addition, when a subgroup analysis of male and female was conducted, the male participants of the treatment group showed a significant reduction at 8th week compared to the baseline (baseline→8the week: 331.59±65.83→310.70±68.49 cm², a reduction of 6.36%).

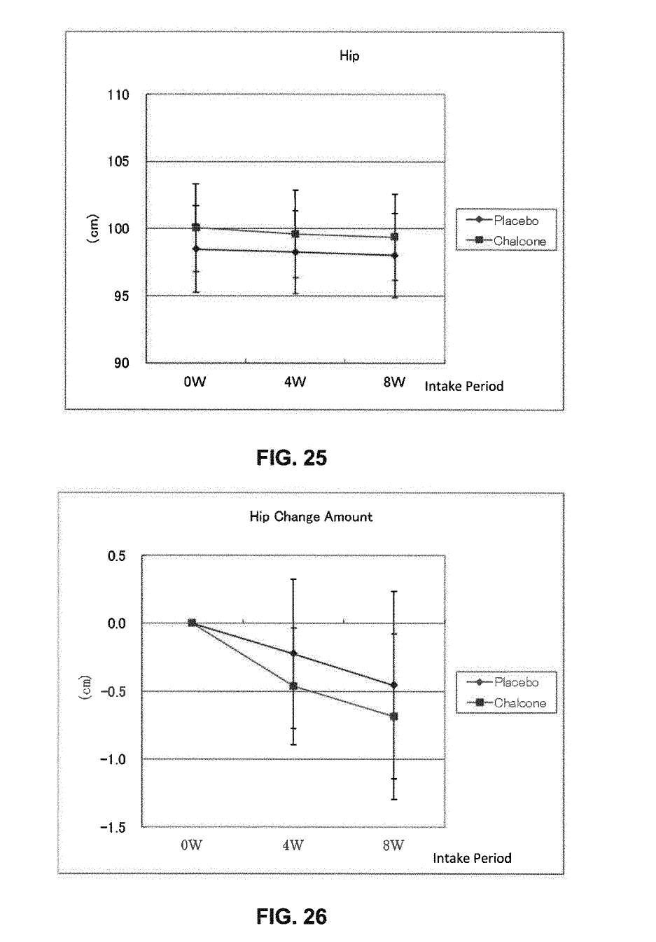

The mean±standard deviation of the hip circumference and the W/H are shown in Table 8. The change amount and the change rate are shown in Tables 9 and 10. The transition is shown in FIGS. 25-26.

TABLE 9
Body Measurement (Change Rate)

| | | | | Num- | | |
|---|---|---|---|---|---|---|
| Item | Unit | Sex | Food | ber | 4th week | 8th week |
| Body | (kg) | All | P | 13 | −0.27 ± 1.09 | −0.22 ± 1.11 |
| weight | | | A | 13 | −0.72 ± 0.91 | −0.82 ± 0.76 |
| | | Male | P | 9 | −0.20 ± 0.75 | −0.21 ± 0.83 |
| | | | A | 9 | −0.82 ± 1.06 | −0.70 ± 0.70 |
| | | Female | P | 4 | −0.43 ± 1.79 | −0.25 ± 1.75 |
| | | | A | 4 | −0.50 ± 0.52 | −1.08 ± 0.92 |
| BMI | | All | P | 13 | −0.11 ± 0.43 | −0.08 ± 0.43 |
| | | | A | 13 | −0.25 ± 0.33 | −0.28 ± 0.28 |
| | | Male | P | 9 | −0.08 ± 0.26 | −0.09 ± 0.27 |
| | | | A | 9 | −0.27 ± 0.37 | −0.21 ± 0.24 |
| | | Female | P | 4 | −0.18 ± 0.73 | −0.05 ± 0.74 |
| | | | A | 4 | −0.20 ± 0.23 | −0.43 ± 0.36 |
| Body | (%) | All | P | 13 | −0.48 ± 0.95 | −0.72 ± 0.57 |
| fat | | | A | 13 | −0.42 ± 1.05 | −0.78 ± 1.34 |
| ratio | | Male | P | 9 | −0.69 ± 1.05 | −0.63 ± 0.34 |
| | | | A | 9 | −0.29 ± 1.07 | −0.93 ± 1.34 |
| | | Female | P | 4 | −0.02 ± 0.54 | −0.93 ± 0.96 |
| | | | A | 4 | −0.73 ± 1.09 | −0.42 ± 1.46 |
| Waist | (cm) | All | P | 13 | −0.73 ± 0.75 | −0.73 ± 0.92 |
| | | | A | 13 | −0.58 ± 0.49 | −1.12 ± 0.73 |
| | | Male | P | 9 | −0.76 ± 0.39 | −0.78 ± 0.68 |
| | | | A | 9 | −0.66 ± 0.57 | −1.08 ± 0.69 |
| | | Female | P | 4 | −0.68 ± 1.36 | −0.63 ± 1.45 |
| | | | A | 4 | −0.40 ± 0.22 | −1.23 ± 0.90 |
| Hip | (cm) | All | P | 13 | −0.22 ± 0.55 | −0.45 ± 0.69 |
| | | | A | 13 | −0.46 ± 0.43 | −0.68 ± 0.61 |
| | | Male | P | 9 | −0.30 ± 0.55 | −0.54 ± 0.64 |
| | | | A | 9 | −0.46 ± 0.43 | −0.50 ± 0.32 |
| | | Female | P | 4 | −0.05 ± 0.60 | −0.25 ± 0.86 |
| | | | A | 4 | −0.48 ± 0.50 | −1.10 ± 0.94 |
| W/H | | All | P | 13 | −0.005 ± 0.007 | −0.003 ± 0.007 |
| | | | A | 13 | −0.001 ± 0.006 | −0.005 ± 0.007 |
| | | Male | P | 9 | −0.005 ± 0.005 | −0.003 ± 0.006 |
| | | | A | 9 | −0.002 ± 0.007 | −0.006 ± 0.007 |
| | | Female | P | 4 | −0.007 ± 0.010 | −0.004 ± 0.010 |
| | | | A | 4 | 0.000 ± 0.005 | −0.002 ± 0.006 |

Value is the mean ± standard deviation.
P: Placebo,
A: Chalcone
*: p < 0.05,
**: p < 0.01 (One sample t-test comparing baseline),
#: p < 0.05,
##: p < 0.01 (two sample t-test comparing placebo group and chalcone group)

TABLE 10
Body Measurement (Change Rate)

| Item | Sex | Food | Number | 4th week (%) | 8th week (%) |
|---|---|---|---|---|---|
| Body weight | All | P | 13 | 99.63 ± 1.55 | 99.70 ± 1.57 |
| | | A | 13 | 99.04 ± 1.20 | 98.89 ± 1.08 |
| | Male | P | 9 | 99.74 ± 0.97 | 99.72 ± 1.06 |
| | | A | 9 | 98.94 ± 1.37 | 99.10 ± 0.94 |
| | Female | P | 4 | 99.38 ± 2.66 | 99.64 ± 2.61 |
| | | A | 4 | 99.26 ± 0.78 | 98.40 ± 1.37 |
| BMI | All | P | 13 | 99.58 ± 1.61 | 99.69 ± 1.60 |
| | | A | 13 | 99.08 ± 1.25 | 98.96 ± 1.07 |
| | Male | P | 9 | 99.72 ± 0.99 | 99.67 ± 1.01 |
| | | A | 9 | 99.02 ± 1.42 | 99.20 ± 0.89 |
| | Female | P | 4 | 99.28 ± 2.75 | 99.74 ± 2.75 |
| | | A | 4 | 99.23 ± 0.89 | 98.40 ± 1.37 |
| Body fat | All | P | 13 | 98.22 ± 3.59 | 97.43 ± 1.78 |
| ratio | | A | 13 | 98.66 ± 3.67 | 97.29 ± 4.74 |
| | Male | P | 9 | 97.41 ± 3.97 | 97.59 ± 1.16 |
| | | A | 9 | 98.94 ± 4.02 | 96.52 ± 4.91 |
| | Female | P | 4 | 100.03 ± 1.78 | 97.06 ± 2.98 |
| | | A | 4 | 98.01 ± 3.15 | 99.01 ± 4.45 |
| Waist | All | P | 13 | 99.21 ± 0.82 | 99.21 ± 0.99 |
| | | A | 13 | 99.40 ± 0.50 | 98.81 ± 0.77 |
| | Male | P | 9 | 99.19 ± 0.42 | 99.17 ± 0.73 |
| | | A | 9 | 99.32 ± 0.58 | 98.87 ± 0.73 |
| | Female | P | 4 | 99.27 ± 1.48 | 99.32 ± 1.58 |
| | | A | 4 | 99.57 ± 0.24 | 98.67 ± 0.95 |
| Hip | All | P | 13 | 99.78 ± 0.56 | 99.54 ± 0.71 |
| | | A | 13 | 99.54 ± 0.43 | 99.32 ± 0.60 |
| | Male | P | 9 | 99.70 ± 0.55 | 99.45 ± 0.65 |
| | | A | 9 | 99.54 ± 0.43 | 99.50 ± 0.32 |
| | Female | P | 4 | 99.95 ± 0.62 | 99.75 ± 0.90 |
| | | A | 4 | 99.53 ± 0.49 | 98.92 ± 0.92 |

Value is the mean ± standard deviation.
P: Placebo,
A: Chalcone
#: p < 0.05,
##: p < 0.01 (two sample t-test comparing placebo group and chalcone group)

The hip circumference decreased at 4th and 8th week in both groups. It was significant at 8^{th} week in the placebo group (Baseline→8th week: 98.48±3.22→98.02±3.14 cm, a reduction of 0.46 cm at 8th week) and at 4th and 8th week in the treatment group (baseline→4th week→8th week: 100.07±3.29→99.61±3.23→99.38±3.20 cm, a reduction of 0.69 cm at 8^{th} week). The W/H decreased at 4th and 8th week in both group. It was significant at 4th week in the placebo group and at 8th week in the treatment group.

The results of PAI-1 (plasminogen activator inhibitor-1), which is used as a risk factor for thrombosis and atherosclerosis, was measured at two facilities, are presented, but neither showed a significant change.

ECLT (euglobulin clot lysis time) significantly extended at 8th week only in the placebo group. The result excluding the samples showing aggregation or cloudiness is also shown, but, like the previous analysis, it significantly extended at 8th week only in the treatment group. However, the group difference was not observed.

tPA activity of the treatment group compared to the placebo group decreased, but none of the differences between and within groups approached statistical significance.

There was no significant change in adiponectin.

Leptin significantly decreased at 8th week in both group. Although the significant difference between groups was not observed, the reduction of 30.9% in the treatment group was greater than that of the placebo group of 14.4%.

Resistin significantly decreased at 4th and 8th week in both groups and the rate of reduction (i.e., the change rate) was almost the same.

In the primary end points, namely, the visceral fat area and body weight, the significant difference between the placebo group and the treatment group was not observed, but only the treatment group showed the significant reduction in the visceral fat area at 8th week and in the body weight at 4th and 8th week.

A significant group difference was not observed in BMI, a secondary end point, but the reduction of the treatment group at 4th and 8th week compared to the baseline were significant. Both groups showed the significant reduction of waist circumference at 4th and 8th week after the intake, but the group difference was not observed. The body fat ratio of both groups decreased at 4th and 8th week, but it was significant only in the placebo group at 8th week compared to the baseline.

Regarding other end points, the total fat area of both groups decreased at 8th week, but only the treatment group showed a significant reduction. There was a significant reduction of the hip circumference in the placebo group at 8th week and in the treatment group at 4th and 8th group.

In the analysis of PAI-1 antigen, ECLT, tPA activity, adiponectin, leptin and resistin, the significant extension of ECLT of the treatment group at 8th week, the significant reduction of leptin of both groups at 8th week and the significant decrease of resistin of both group were observed.

From the result above, the significant reduction of the visceral fat area and the body weight were confirmed only in the treatment group although the difference between groups was not confirmed.

Therefore, the reduction effect on the visceral fat area and the body weight by the intake of Ashitaba chalcone were shown in the studies.

The mean of the heart rate significantly increased at 4th week in the treatment group compared to the baseline. However, it was a mild change and was not clinically a problem.

Any other significant changes were not confirmed.

Compared to the baseline, the total cholesterol and the LDL-cholesterol significantly increased at 4th week in the placebo group. However, it was a small change and not clinically significant.

There was a reduction of ALP in the placebo group at 4th and 8th week compared to the baseline, but not a clinical problem.

There was a significant change compared to the baseline in the total protein (a reduction at 4th and 8th week in the placebo group and at 4th week in the treatment group), albumin (a reduction at 4th week in the placebo group) and creatinine (an increase at 8^{th} week). However, these were small and not clinically problems.

There was no significant change was observed in the glucose and HbAlc.

Na and K showed a significant increase at 8th week in the treatment group compared to the baseline, but it was a small change and not clinically a problem.

There was a significant change compared to the baseline in the white blood cell count (a reduction at 4th week in the placebo group and at 4th and 8th week in the treatment group), MCV (mean corpuscular volume) (an increase at 4th and 8th week in the placebo group and at 8th week in the treatment group), MCH (mean corpuscular hemoglobin) (an increase at 8th week in both groups), MCHC (mean corpuscular hemoglobin concentration) (a reduction at 4th and 8th week) and the blood platelet (a reduction at 4th and 8th week in the placebo group).

Each change was small and not clinically a problem.

The urinary protein, urine sugar and urine occult blood reaction were all measured. There was no problem confirmed.

There was a significant change in some test items (namely, the total cholesterol, LDL-cholesterol, ALP, the total protein, albumins, creatinine, the white blood cell count, MCV, MCH, MCHC and the platelet count in the placebo group and the pulse rate, the total protein, Na, K, the white blood cell count, MCV and MCH in the treatment group), but any of the change was small and not clinically significant. Taking the above into consideration, it was determined that there is no safety issue in this study where 200 mg of Ashitaba chalcone was taken for 8 weeks.

The intake of energy, protein, fat and carbohydrates for 3 days before each test were computed.

There was no significant change of the energy intake in both groups. The protein intake at 8^{th} week after the start of the intake significantly increased at 8th week in the all cases and the male participants of the treatment group. A significant increase of the fat intake at 4th week in the male participants of the treatment group was observed. Any significant change of the carbohydrate intake was observed in both groups.

Each change in the treatment group was small and not an issue in the analysis of the evaluation items.

In this study, where slightly obese adult males and females took a test food containing 200 mg of Ashitaba chalcone powder for 8 weeks, a significant difference between groups was not observed but a significant reduction of the visceral fat area and body weight was confirmed only in the treatment group compared to the baseline.

When the reduction of the visceral fat was compared to that of the subcutaneous fat in the current study on the intake of Ashitaba chalcone, the former was large in the male participants and the latter was, although the sample is small and no significant difference was observed, large in the female participants. It is argued, although it is a subsequent research topic, that the body fat decreases by the same or similar mechanism to that of cellulite solution and that the body weight reducing effects are present.

In the slightly obese male and female participants who took the test food containing 200 mg of Ashitaba chalcone for 8 weeks (i.e., the treatment group), the significant decrease of the visceral fat area, total fat area and the body weight (BMI) were confirmed.

There was no issue in the safety when the test food containing 200 mg of Ashitaba chalcone as the principal ingredient was taken for 8 weeks.

The significant reduction of the visceral fat area and body weight were confirmed only in the treatment group so that the visceral fat-reducing effect by the intake of Ashitaba chalcone can be expected.

### Example 5

### Effect of Chalcone on Visceral Fat, Body Composition, Lipids, Energy Levels and Control of Blood Sugar

This purpose of this study was to determine if the chalcone-based supplement when administered as directed shows effect on visceral fat, body composition, lipids, energy levels and aid in the control of blood sugar in adults with metabolic syndrome. This beneficial effect has been demonstrated in a pre-clinical study.

A total of N=60 male and female participants (n=30 male, n=30 female) were randomized in in a 1:1 ratio of Active Product to Placebo. Subjects were asked to come to the study center for a screening visit and for 4 study visits.

The clinical study lasted approximately 84 days (12 weeks) with subjects receiving either active product or placebo for the study duration.

All eligible subjects were scheduled for In-Clinic Study Visits at Visit 1 (Screening; Day −60 to Day −1), Visit 2 (Randomization/Baseline; Day 1), Visit 3 (Day 28), Visit 4 (Day 56), and Visit 5 (EOS; Day 84).

Table 11 summarizes the number of days between the five visits for the safety and efficacy populations respectively. One subject (Subject #129, placebo group, male) dropped from the study after Visit 2.

The visit time intervals were consistent with the requirements of the protocol, which were within +/−5 days from the planned visit interval. The time interval from Day 1 (Baseline) to Day 28 (Visit 3) was 27 days for all subjects.

TABLE 11
Intervals (Days) Between Visits

| Day 0 (Screen) | Day 1 (Baseline) | Day 28 (Visit 3) | Day 56 (Visit 4) |
|---|---|---|---|
| to Day 1 | to Day 28 | to Day 56 | to Day 84 |
| (Baseline) | (Visit 3) | (Visit 4) | (Visit 5) |
| 31.4 ± 2.6 (60) | 27.0 ± 0.0 (59) | 27.8 ± 0.05 (59 | 28.2 ± 0.05 (59) |
| 16.00 (4-58) | 27 (27-27) | 28.0 (27.0-28.0) | 28.0 (28-29) |

Note.
Values presented as:
Mean ± Standard Error (N)
Median (Minimum-Maximum)

The screening/baseline characteristics variables are reported in Table 12. The variables are summarized in the format:

Mean±Standard Error (Sample Size)

Median (Minimum−Maximum

EOS measurements and p-values from a paired t-test comparing baseline and EOS means for vital signs only (blood pressure and heart rate) are included in the table. Significant differences were found from screening to EOS heart rate. The mean heart rate decreased from screening (M=79.1, SE=11.7) to EOS (M=73.7, SE=10.2).

No statistically significant difference between the groups (mean differences between product and placebo) was observed for the visceral fat measurement at Day 1 (baseline), Day 56 or Day 84 (EOS), or for changes from screening. No statistically significant difference for either group was observed over the testing period.

No statistically significant difference between the groups (mean differences between product and placebo) was observed for the visceral fat measurement at Day 1 (baseline), Day 56 or Day 84 (EOS), or for changes from screening. No statistically significant difference within the groups was observed for either group across time. A trending significance (p≧0.05, but <0.10) was noted for the following endpoints:

Product Group:
Decrease in visceral fat from Day 1 to Day 56 (p=0.080), with a large effect size.
Decrease in visceral fat from Day 1 to Day 84 (p=0.069), with a large effect size.

Placebo Group:
Decrease in visceral fat from Day 1 to Day 56 (p=0.095), with a large effect size.

No statistically significant difference between the groups (mean differences between product and placebo) was observed for the visceral fat measurement at Day 1 (baseline), Day 56 or Day 84 (EOS), or for changes from screening. No statistically significant difference within the groups was observed for either group across time. A trending significance (p≧0.05, but <0.10) was noted for the following endpoints:

Product Group:
Increase in visceral fat from Day 1 to Day 56 (p=0.052), with a large effect size.

No statistically significant difference between the groups (mean differences between product and placebo) was observed for the body fat measurement at Day 1 (baseline) or Day 84 (EOS), or for changes from screening. No statistically significant difference within the groups was observed for either group across time.

A significant difference between the groups (mean differences between product and placebo) was observed at Day 84 (EOS; p=0.047). Body fat % was significantly greater for product (M=26.1, SE=0.6) than for placebo (M=24.1, SE=1.0). No statistically significant difference within the groups was observed for either group across time.

No statistically significant difference between the groups (mean differences between product and placebo) was observed for the body fat measurement at Day 1 (baseline) or Day 84 (EOS), or for changes from screening. No statistically significant difference within the groups was observed for either group across time.

No statistically significant difference between the groups (mean differences between product and placebo) was observed for the fat mass measurement at Day 1 (baseline) or Day 84 (EOS), or for changes from screening. No statistically significant difference within the groups was observed for either group across time.

No statistically significant difference between the groups (mean differences between product and placebo) was observed for the fat mass measurement at Day 1 (baseline) or Day 84 (EOS), or for changes from screening. No statistically significant difference within the groups was observed for either group across time.

No statistically significant difference between the groups (mean differences between product and placebo) was observed for the fat mass measurement at Day 1 (baseline) or Day 84 (EOS), or for changes from screening. No statistically significant difference within the groups was observed for either group across time.

No statistically significant difference between the groups (mean differences between product and placebo) was observed for the fat free mass measurement at Day 1 (baseline) or Day 84 (EOS), or for changes from screening. No statistically significant difference within the groups was observed for either group across time.

No statistically significant difference between the groups (mean differences between product and placebo) was observed for the fat free mass measurement at Day 1 (baseline) or Day 84 (EOS), or for changes from screening. No statistically significant difference within the groups was observed for either group across time.

No statistically significant difference between the groups (mean differences between product and placebo) was observed for the fat free mass measurement at Day 1 (baseline) or Day 84 (EOS), or for changes from screening. No statistically significant difference within the groups was observed for either group across time. A trending significance (p≧0.05, but <0.10) was noted for the following endpoints:

Product group:
Decrease in fat free mass from Day 1 to Day 84 (p=0.098), with a large effect size.

No statistically significant difference between the groups (mean differences between product and placebo) was observed for the total cholesterol measurement at Day 0 (screening) or Day 84 (EOS), or for changes from screening. No statistically significant difference within the groups was observed for either group across time.

A statistically significant difference between the groups (mean differences between product and placebo) was observed for total cholesterol at Day 0 (screening; p=0.022) and Day 84 (EOS; p=0.014). At both times, product was significantly greater than placebo. The ANCOVA analysis included the screening measurement as a covariate and thus adjusted for differences between the two groups at screening. The result of the ANCOVA analysis was not significant, indicating no differences between the two groups at Day 84 (EOS) after controlling for the screening measurement. No statistically significant difference within the groups was observed for either group across time.

The subjects in the placebo group had significantly higher total cholesterol than the subjects in the product group at Day 0, before any treatment began. Subjects in the placebo group also had significantly higher cholesterol than subjects in the product group at Day 84. However, when the measurements for total cholesterol at Day 84 were adjusted for (a) the total cholesterol measurements at Day 0, (b) BMI, and (c) gender, the change in total cholesterol from Day 0 to Day 84 was not significantly different between product and placebo.

No statistically significant difference between the groups (mean differences between product and placebo) was observed for the total cholesterol measurement at Day 0 (screening) or Day 84 (EOS), or for changes from screening. No statistically significant difference within the groups was observed for either group across time. A trending significance (p≧0.05, but <0.10) was noted for the following endpoint:

Between Groups (Product vs. Placebo):
Subjects in the placebo group had a higher total cholesterol measurement at Day 84 than did subjects in the product group (p=0.095). The effect size was large.

A statistically significant difference between the groups (mean differences between product and placebo) was observed for triglyceride levels at Day 0 (screening; p=0.033) and Day 84 (EOS; p=0.002). At both times, product was significantly greater than placebo. The ANCOVA analysis included the screening measurement as a covariate and thus adjusted for differences between the two groups at screening. The result of the ANCOVA analysis was significant (p=0.036), indicating statistically significant differences remained between the two groups at Day 84 (EOS) after controlling for the screening measurements. And product was significantly greater than placebo.

Even after adjusting for (a) the baseline measurements of triglyceride levels, (b) BMI, and (c) gender, the average value of triglyceride levels at Day 84 was still significantly greater for product than placebo.

A statistically significant difference between the groups (mean differences between product and placebo) was observed for triglyceride levels at Day 0 (screening; p=0.007) and Day 84 (EOS; p<0.001). At both times, product was significantly greater than placebo. The ANCOVA analysis included the screening measurement as a covariate and thus adjusted for differences between the two groups at screening. The result of the ANCOVA analysis was significant (p=0.029), indicating statistically significant differences remained between the two groups at Day 84 (EOS) after controlling for the screening measurements. And product was significantly greater than placebo.

Even after adjusting for (a) the baseline measurements of triglyceride levels and (b) BMI, the average value of triglyceride levels at Day 84 was still significantly greater for product than placebo. These findings are similar to the triglyceride measurements for all records (see Table 4.4.6a, p. 67)

A statistically significant difference between the groups (mean differences between product and placebo) was observed for triglyceride levels in the change between Day 0 (screening) and Day 84 (EOS; p=0.048). Product (M_{change}=38.2, SE_{change}=12.7) had a significantly greater increase in measurement over time than placebo (M_{change}=5.5, SE_{change}=14.1). The ANCOVA analysis included the screening measurement as a covariate and thus adjusted for differences between the two groups at screening. The result of the ANCOVA analysis was not significant (p=0.234), indicating no differences between the two groups at Day 84 (EOS) after controlling for the screening measurements and BMI

After adjusting for (a) the baseline measurements of triglyceride levels and (b) BMI, the average value of triglyceride levels at Day 84 was no longer significantly different between product and placebo groups.

However, for the product group only, a statistically significant difference within the group was observed across time. The product group had statistically significant increases in triglyceride levels from Day 0 (M=137.4, SE=13.9) to Day 84 (M=175.6, SE=21.0).

No statistically significant difference between the groups (mean differences between product and placebo) was observed for the LDL measurement at Day 0 (screening) or Day 84 (EOS), or for changes from screening. No statistically significant difference within the groups was observed for either group across time.

A statistically significant difference between the groups (mean differences between product and placebo) was observed for the LDL measurement at Day 0 (screening; p=0.022). The LDL measurement was significantly greater for the product group (M=147.7, SE=6.2) than for the placebo group (M=127.7, SE=7.2). The ANCOVA analysis included the screening measurement and BMI as covariates and thus adjusted for any differences between the two groups at screening. The result of the ANCOVA analysis was not significant (p=0.700), indicating no differences between the two groups at Day 84 (EOS) after controlling for the screening measurements.

Although there were differences in the average measurement of LDL between product and placebo groups at Day 0, there were not significant differences between product and placebo at Day 84, and no differences between product and placebo at Day 84 after adjusting for (a) the LDL measurement at Day 0 and (b) BMI. No statistically significant difference within the groups was observed for either group across time.

No statistically significant difference between the groups (mean differences between product and placebo) was observed for the LDL measurement at Day 0 (screening) or Day 84 (EOS), or for changes from screening. No statistically significant difference within the groups was observed for either group across time. A trending significance (p≧0.05, but <0.10) was noted for the following endpoints:

Between Groups (Product vs. Placebo):
Subjects in the placebo group had a higher LDL measurement at Day 84 than did subjects in the product group (p=0.056). The effect size was large.

No statistically significant difference between the groups (mean differences between product and placebo) was observed for the HDL measurement at Day 0 (screening) or Day 84 (EOS), or for changes from screening. A statistically significant difference within the groups was observed for the product group across time (p=0.001). The HDL for the product group significantly decreased from Day 0 (M=47.1, SE=2.3) to Day 84 (M=44.1, SE=2.1). A trending significance (p>0.05, but <0.10) was noted for the following endpoints:

Placebo Group:
Decrease in HDL from Day 1 to Day 84 (p=0.095), with a large effect size.

No statistically significant difference between the groups (mean differences between product and placebo) was observed for the HDL measurement at Day 0 (screening) or Day 84 (EOS), or for changes from screening. No statistically significant difference within the groups was observed for either group across time. A trending significance (p≧0.05, but <0.10) was noted for the following endpoints:

Between Groups (Product vs. Placebo):
Subjects in the placebo group had a higher HDL measurement at Day 84 than did subjects in the product group (p=0.060). The effect size was small.

No statistically significant difference between the groups (mean differences between product and placebo) was observed for the HDL measurement at Day 0 (screening) or Day 84 (EOS), or for changes from screening. A statistically significant difference within the groups was observed for the product group across time (p=0.002). The HDL for the product group significantly decreased from Day 0 (M=55.1, SE=3.2) to Day 84 (M=50.7, SE=3.3). A statistically significant difference within the groups was observed for the placebo group across time (p=0.016). The HDL for the placebo group significantly decreased from Day 0 (M=54.0, SE=3.8) to Day 84 (M=49.7, SE=3.4).

No statistically significant difference between the groups (mean differences between product and placebo) was observed for the POMS-VA measurement at Day 1 (baseline) or Day 84 (EOS), or for changes from baseline. No statistically significant difference within the groups was observed for either group across time.

No statistically significant difference between the groups (mean differences between product and placebo) was observed for the POMS-VA measurement at Day 1 (baseline) or Day 84 (EOS), or for changes from baseline. No statistically significant difference within the groups was observed for either group across time. A trending significance (p≧0.05, but <0.10) was noted for the following endpoints:

Placebo Group:
Increase in POMS-VA from Day 1 to Day 84 (p=0.063), with a large effect size.

No statistically significant difference between the groups (mean differences between product and placebo) was observed for the POMS-VA measurement at Day 1 (baseline) or Day 84 (EOS), or for changes from baseline. A statistically significant difference within the groups was observed for the product group across time (p=0.023). The POMS-VA for the product group significantly decreased from Day 1 (M=21.5, SE=1.5) to Day 84 (M=17.7, SE=1.5). A trending significance (p≧0.05, but <0.10) was noted for the following endpoints:

Placebo Group:
Decrease in POMS-VA from Day 1 to Day 84 (p=0.067), with a large effect size.

No statistically significant difference between the groups (mean differences between product and placebo) was observed for the POMS-FI measurement at Day 1 (baseline) or Day 84 (EOS), or for changes from baseline. No statistically significant difference within the groups was observed for either group across time.

A statistically significant difference between the groups (mean differences between product and placebo) was observed for the POMS-FI measurement at Day 1 (baseline). The POMS-FI score was significantly higher for the product group (M=6.6, SE=1.6) than for the placebo group (M=2.9, SE=0.8; p=0.028). Significant differences were not found between product and placebo at or Day 84 (EOS). A statistically significant difference within the groups was observed for the product group across time (p=0.048). The POMS-FI measurement for the product group significantly decreased from Day 1 (M=6.6, SE=1.6) to Day 84 (M=3.9, SE=1.4).

Although the two groups of product vs. placebo were significantly different at baseline, the average change from baseline between product and placebo was not significantly different in the ANCOVA analysis. This is because ANCOVA adjusted for (a) the baseline measurement of POMS-FI and (b) BMI when calculating differences between product and placebo at Day 84. A trending significance (p>0.05, but <0.10) was noted for the following endpoints:

Between Groups (Product vs. Placebo):
Subjects in the placebo group had a higher HDL measurement at Day 84 than did subjects in the product group (p=0.060). The effect size was small.

However, the ANCOVA was not significant or trending for differences between product and placebo at Day 84 (p=0.426) which means the treatment effect was not significant after controlling for baseline POMS-FI and BMI.

A statistically significant difference between the groups (mean differences between product and placebo) was observed for the POMS-FI measurement for the change from Day 1 (p=0.041). The mean change in the POMS-FI measurement was significantly greater for the placebo group (M=−3.0, SE=1.0) than for the placebo group (M=0.1, SE=1.4). In other words, POMS-FI measurement decreased on average 3 points for the placebo group, but in essence remained unchanged for the product group. The ANCOVA analysis included the screening measurement as a covariate and thus adjusted for (a) the baseline measurement of POMS-FI and (b) BMI. The result of the ANCOVA analysis was not significant (p=0.139), indicating no differences between the two groups at Day 84 (EOS) after controlling for the baseline measurements and BMI.

Although the t-test was significant for the change from baseline to measurements at Day 84, the ANCOVA test, which adjusted for differences in baseline POMS-FI measurement and BMI, indicated that the change from baseline was not statistically significant between the product and placebo groups, thus not different.

A statistically significant difference within the groups was observed for the placebo group across time (p=0.008). The POMS-FI for the product group significantly decreased from Day 1 (M=5.7, SE=1.1) to Day 84 (M=2.7, SE=0.7).

No statistically significant difference between the groups (mean differences between product and placebo) was observed for the HgBA1c measurement at Day 0 (screening) or Day 84 (EOS), or for changes from screening. A statistically significant difference within the groups was observed for the placebo group across time (p=0.001). The HgBA1c measurement placebo group significantly increased from Day 0 (M=5.4, SE=0.1) to Day 84 (M=5.6, SE=0.1).

No statistically significant difference between the groups (mean differences between product and placebo) was observed for the HgBA1c measurement at Day 0 (screening) or Day 84 (EOS), or for changes from screening. No statistically significant difference within the groups was observed for either group across time.

No statistically significant difference between the groups (mean differences between product and placebo) was observed for the HgBA1c measurement at Day 0 (screening) or Day 84 (EOS), or for changes from screening. A statistically significant difference within the groups was observed for the product group across time (p=0.003). The HgBA1c measurement for the product group significantly increased from Day 0 (M=5.3, SE=0.1) to Day 84 (M=5.5, SE=0.1). A statistically significant difference within the groups was observed for the placebo group across time (p=0.005). The HgBA1c measurement for the placebo group significantly increased from Day 0 (M=5.3, SE=0.1) to Day 84 (M=5.6, SE=0.1).

A statistically significant difference between the groups (mean differences between product and placebo) was observed for the ghrelin measurement for the change from Day 1 (p=0.019). The mean ghrelin measurement for the placebo group increased (M_{change}=58.58, SE_{change}=32.17), but decreased for the product group (M_{change}=−34.37, SE_{change}=29.72). The ANCOVA analysis included the screening measurement as a covariate and thus adjusted for differences between the two groups at screening. The result of the ANCOVA analysis was not significant (p=0.062), indicating no differences between the two groups at Day 84 (EOS) after controlling for the baseline measurements. However, of note is a trending significance for the ANCOVA analysis (see below)

There was a significant difference in the average change from baseline between product and placebo groups. Ghrelin for subjects in the product group decreased about 34 (pg/ml) but ghrelin for the subjects in the placebo group increased on average about 59 (pg/ml). The ANCOVA analysis adjusted for (a) the baseline measurement of ghrelin and (b) BMI, and (c) gender and tests the outcome of ghrelin at Day 84. Although the ANCOVA was not statistically significant, it trended towards significance (see trending significance, between groups, below). No statistically significant difference within the groups was observed for either group across time. A trending significance (p>0.05, but <0.10) was noted for the following endpoints:

Between Groups (Product vs. Placebo):
For the ANCOVA test, which adjusted for baseline ghrelin measurements and BMI, and gender, subjects in the placebo group had a higher ghrelin measurement at Day 84 than did subjects in the product group (p=0.062). The effect size was small to moderate.

Placebo Group:
Increase in ghrelin from Day 1 to Day 84 (p=0.079), with a large effect size.

No statistically significant difference between the groups (mean differences between product and placebo) was observed for the ghrelin measurement at Day 0 (screening) or Day 84 (EOS), or for changes from screening. No statistically significant difference within the groups was observed for either group across time.

No statistically significant difference between the groups (mean differences between product and placebo) was observed for the ghrelin measurement at Day 0 (screening) or Day 84 (EOS), or for changes from screening. No statistically significant difference within the groups was observed for either group across time. A trending significance (p≧0.05, but <0.10) was noted for the following endpoints:

Between Groups (Product vs. Placebo):

A trend towards significance for mean differences between product and placebo was observed for the ghrelin measurement for Day 1 (p=0.068) and for change from Day 1 to Day 84 (p=0.053).

Most likely any observed trends towards significance are observed because of the difference in baseline measurements between product and placebo. When controlling for baseline and BMI, the ANCOVA model indicted that the average ghrelin measurement for product and placebo were not different at Day 84. From the table, one can see that the ghrelin measurement at baseline was much higher than placebo, but that at Day 84 the measurements were much closer in value.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

What is claimed is:

1. A composition for weight loss comprising:
a) at least 8% total chalcones comprising 4-hydroxyderricin and xanthoangelol; and
b) coumarins.

2. The composition of claim 1, wherein the coumarins are selected from a group comprising furanocoumarin type coumarins and dihydropyrano-coumarins.

3. The composition of claim 2, wherein the furanocoumarins are selected from a group comprising xanthotoxin (psoralen), isopimpinellin, bergapten, and imperatorin.

4. The composition of claim 2, wherein the dihydropyrano-coumarins are selected from a group comprising laserpitin, isolaserpitin, and selinidin.

5. The composition of claim 1, wherein the amounts of coumarins comprises approximately 35 mg/g to 45 mg/g.

6. A composition for reduction of visceral fat comprising:
a) at least 8% total chalcones comprising 4-hydroxyderricin and xanthoangelol; and
b) coumarins.

7. The composition of claim 6, wherein the coumarins are selected from a group comprising furanocoumarin type coumarins and dihydropyrano-coumarins.

8. The composition of claim 7, wherein the furanocoumarins are selected from a group comprising xanthotoxin (psoralen), isopimpinellin, bergapten, and imperatorin.

9. The composition of claim 7, wherein the dihydropyrano-coumarins are selected from a group comprising laserpitin, isolaserpitin, and selinidin.

10. The composition of claim 6, wherein the amounts of coumarins comprises approximately 35 mg/g to 45 mg/g.

11. A method to isolate and purify chalcones.

12. A method for inducing weight loss comprising administration of an effective amount of chalcone powder obtained by the method of claim 11.

13. A method for inducing reduction of visceral fat comprising administration of an effective amount of chalcone powder obtained by the method of claim 11.

14. The method of claim 11 comprising the steps of:
a) cutting a stem of an Ashitaba plant;
b) harvesting sap from the cut stem;
c) pasteurizing the sap;
d) mixing the pasteurized sap with cyclodextrin to produce a mixture;
e) sterilizing the mixture;
f) freeze-drying the mixture; and
g) pulverizing the mixture.

15. The method of claim 14, wherein the step of pasteurizing the sap comprises heating the sap at 90° C. for 30 minutes.

16. The method of claim 14, wherein the step of sterilizing the mixture comprises heating the mixture at 121° C. for 15 minutes.

17. The method of claim 14, wherein the step of pulverizing comprises passing the mixture through a 100 mesh.
